(19) United States
(12) Patent Application Publication
(10) Pub. No.: US 2024/0050915 A1
(43) Pub. Date: Feb. 15, 2024

(54) MICROBIAL MICROCAPSULE AND METHOD FOR PRODUCING SAME

(71) Applicant: KAO CORPORATION, Tokyo (JP)

(72) Inventors: Satoshi SUGAHARA, Kamisu-shi (JP); Kenichi SHIKATA, Kamisu-shi (JP); Yu SAKURAI, Kamisu-shi (JP)

(73) Assignee: KAO CORPORATION, Tokyo (JP)

(21) Appl. No.: 18/259,219

(22) PCT Filed: Dec. 24, 2021
(86) PCT No.: PCT/JP2021/048212

§ 371 Date: Jun. 23, 2023

(30) Foreign Application Priority Data
Dec. 24, 2020 (JP) 2020-215262

(51) Int. Cl. B01J 13/08; C12N 1/16; C12N 1/12

(52) U.S. Cl. CPC B01J 13/08; C12N 1/16; C12N 1/12

(57) ABSTRACT

A method for producing a microbial microcapsule, including, mixing a (A) hydrophobic component having a surface tension above 33.6 mN/m at 25° C. and a microorganism (B) for 3 hours or more. The mixing is carried out under a condition that a mass ratio of the (A) hydrophobic component to a dry mass of the microorganism (B), [(A)/(B)], is above 2.

### FIELD OF THE INVENTION

The present invention relates to a microbial microcapsule and a method for producing the same.

### BACKGROUND OF THE INVENTION

Microcapsules are micrometer-sized microcapsules composed of a core material and a membrane containing the core material. Microcapsules containing flavoring agents, pharmaceuticals, agricultural chemicals, and the like, encapsulated in capsules composed of a polymer compound as a membrane substrate are industrially utilized for the purpose of, for example, suppressing volatilization of active ingredients and improving delivery efficacy.

Typical methods for producing microcapsules include a spray drying method as a physical method, a coacervation method as a physicochemical method, and an interfacial polymerization method or an in situ polymerization method as a chemical method.

Meanwhile, microbial microcapsules utilizing microorganisms per se as a membrane substrate have been proposed. In addition to their basic performance as capsules, microbial microcapsules have unique functions of biomaterials such as biodegradability, high environmental endurance, water dispersibility, monodispersibility, and eatability of pests.

Cell walls of yeasts are widely utilized in microbial microcapsules; for example, a method of producing a microcapsule in which an enzymatically treated yeast cell is treated with an acid aqueous solution and then a hydrophobic liquid such as oleic acid is encapsulated in the yeast cell (Patent Literature 1), a method of producing a particle in which a terpene emulsion and a suspension of yeast cell wall particles or yeast glucan particles are mixed and incubated to produce a particle in which a terpene component is encapsulated (Patent Literature 2), and the like have been reported.

### CITATIONS

(Patent Literature 1) JP hei 8-243378-A
(Patent Literature 2) JP 2014-28838-A

### SUMMARY OF THE INVENTION

The present invention provides a method for producing a microbial microcapsule, including mixing (A) a hydrophobic component having a surface tension above 33.6 mN/m at 25° C. and (B) a microorganism, wherein the mixing is carried out under a condition of mass ratio of the (A) hydrophobic component to the dry mass of the (B) microorganism, [(A)/(B)], being above 2.

The present invention also provides a microbial microcapsule comprising (A) a hydrophobic component having a surface tension above 33.6 mN/m at 25° C. encapsulated in (B) a microorganism, wherein the microbial microcapsule has an encapsulation percentage above 54 mass % defined by the following equation (1).

Encapsulation percentage (mass %)=[mass of (A) hydrophobic component/(mass of (A) hydrophobic component+dry mass of (B) microorganism)]×100 (1)

### DETAILED DESCRIPTION OF THE INVENTION

However, in the conventional method described above, the respective hydrophobic components are hardly incorporated into the microorganisms, and the obtained microbial microcapsules encapsulates the hydrophobic components merely in a low encapsulation percentage.

Accordingly, the present invention relates to a microbial microcapsule encapsulating a hydrophobic component at a high percentage and a method for producing the same.

As a result of earnest research with focusing on the surface tension of the hydrophobic component to be encapsulated in the microorganism, the present inventors found that the hydrophobic component having at least a certain surface tension can be easily encapsulated in the microorganism, and that in order to encapsulate the hydrophobic component, setting the mix ratio of the microorganism and the hydrophobic component to a certain value or higher can serve to give a microbial microcapsule containing a hydrophobic component in a higher encapsulation percentage than ever.

The present invention facilitates to give a microbial microcapsule encapsulating a large amount of hydrophobic components.

### [Method for Producing Microbial Microcapsule]

The method for producing a microbial microcapsule of the present invention includes mixing (A) a hydrophobic component having a surface tension above 33.6 mN/m at 25° C. and (B) a microorganism, and the mixing is performed under a condition of mass ratio of the (A) hydrophobic component to a dry mass of the (B) microorganism (B), [(A)/(B)], being above 2. Setting the mass ratio of the (A) hydrophobic component to the dry mass of the (B) microorganism, [(A)/(B)], to above 2, the (A) hydrophobic component can be encapsulated in the microorganism (B) at a high encapsulation percentage.

Hereinafter, in the present specification, a step of mixing (A) a hydrophobic component having surface tension above 33.6 mN/m at 25° C. and (B) a microorganism may be also referred to as a mixing step.

In the present specification, the (A) hydrophobic component refers to a hydrophobic component having a surface tension above 33.6 mN/m at 25° C. The (A) hydrophobic component preferably has a surface tension of 35.1 mN/m or more, and more preferably 36.3 mN/m or more at 25° C., from the viewpoint of ease of encapsulation in microorganisms. The upper limit of the surface tension is not particularly limited, but it is preferably 72 mN/m or less (less than or equal to the surface tension of water), and more preferably 38.5 mN/m or less. The surface tension at 25° C. of the (A) hydrophobic component is above 33.6 mN/m, preferably above 33.6 mN/m and 72 mN/m or less, more preferably from 35.1 to 72 mN/m, further more preferably from 35.1 to 38.5 mN/m, and even more preferably from 36.3 to 38.5 mN/m.

Examples of the (A) hydrophobic component include carvacrol (35.1 mN/m), methyl salicylate (36.3 mN/m), and benzyl alcohol (38.5 mN/m). Note that the numerical value in parentheses indicates a surface tension at 25° C.

The hydrophobic component is not particularly limited as long as it is separated from water in a liquid-liquid phase separation manner at the temperature of encapsulation as described hereinafter. The hydrophobic component preferably has a log P value of 1.0 or more, more preferably 1.46 or more, from the viewpoint of the encapsulation percentage of the hydrophobic component, and preferably 30 or less, more preferably 20 or less, and even more preferably 10 or less from the same viewpoint. The log P value is preferably from 1.0 to 30, more preferably from 1.46 to 20, and even more preferably from 1.46 to 10. Log P value of methyl salicylate and benzyl alcohol described above is each 1.46, and log P value of carvacrol is 3.37.

Log P value refers to a common logarithm of the partition coefficient between 1-octanol and water, which is an indicator of hydrophobicity of organic compounds. As this value goes greater in a positive direction, it indicates that the compound has higher hydrophobicity. Log P value of the hydrophobic components are calculated by using Chem Draw 18.2, in which Molecular Networks chemoinformatics platform MOSES based computational modules is used for the computational methods. MOSES is developed, maintained and owned by Molecular Networks GmbH (Erlangen, Germany).

Preferred hydrophobic components include components used in, for example, pharmaceuticals, quasi-drugs, cosmetics, foods, and agrochemicals. Among them, from the viewpoint of utilizing the edible property of pests of microbial microcapsules, insecticidal components for sanitary pests and for agricultural pests are preferred.

The (A) hydrophobic component may be one or a mixture of two kinds or more. When the (A) hydrophobic component is a mixture of two kinds or more, the surface tension at 25° C. of the (A) hydrophobic component means the surface tension as a mixture of the two kinds or more. Accordingly, as long as the surface tension at 25° C. of the two or more mixtures falls in the above ranges, a hydrophobic component having a surface tension at 25° C. of 33.6 mN/m or less by itself may be used in combination.

In the present specification, the surface tension at 25° C. of the (A) hydrophobic component can be measured by the method described in Examples described hereinafter.

In the present specification, the (B) microorganism is not particularly limited, but it is preferably a microorganism having a cell wall for encapsulating more amount of the (A) hydrophobic component, more preferably a yeast, a fine alga, and a filamentous fungus, and even more preferably a yeast and a fine alga.

Examples of the yeast include yeasts such as Saccharomyces genus, Candida genus, Rhodotorula genus, and Pichia genus. Among them, it is preferably a yeast of the genus Saccharomyces, and more preferably a Saccharomyces cerevisiae.

Examples of the fine alga include preferably an alga of the order Eustigmatales, and more preferably an alga of the genus Nannochloropsis. Among them, it is preferably Nannochloropsis oculata, Nannochloropsis oceanica, Nannochloropsis gaditana, Nannochloropsis salina, Nannochloropsis atomus, Nannochloropsis maculata, Nannochloropsis granulata, Nannochloropsis sp., and more preferably Nannochloropsis sp.

The microorganism (B) may be used as long as it can serve as a membrane material, and may be a live state, a dry state, or a dead state.

The shape of the (B) microorganism may be an egg shape, a spherical shape, a lens shape, and an oval shape. The shape is preferably substantially a spherical shape, from the viewpoint of coherence and viscosity. From a similar viewpoint, the diameter of the (B) microorganism is preferably from 0.5 to 30 μm, more preferably from 1 to 20 μm, and even more preferably from 2 to 15 μm. Here, in the present specification, the diameter of the microorganism (B) refers to a median diameter measured by a laser diffraction/scattering Particle Size Distribution Analyzer (LA-920) manufactured by HORIBA, Ltd.

The (B) microorganism is preferably subjected to elution of the intracellular components in advance for encapsulating more amount of the (A) hydrophobic component and improvement of the encapsulation percentage of the (A) hydrophobic component. Examples of the elution of the intracellular components include known methods such as enzymatic treatment. After the enzymatic treatment, another treatment such as an acid treatment may be further performed.

The enzyme used in the enzymatic treatment is preferably at least one selected from the group consisting of an autodigestive enzyme, a protease, a glucanase, a chitinase and a mannase possessed by the microorganism per se. The conditions of the enzymatic treatment are not particularly limited, but the treatment temperature may be from 30 to 60° C., preferably from 40 to 50° C. The treatment time is from 1 to 48 hours, preferably from 15 to 24 hours.

Examples of the acid used in the acid treatment may include inorganic acids such as hydrochloric acid, nitric acid, sulfuric acid and phosphoric acid, and organic acids such as citric acid, lactic acid and ascorbic acid. The condition of the acid treatment is not particularly limited, but the pH may be adjusted to from 0 to 2, preferably 1 or less, and more preferably 0.5 or less by adding an acid. The treatment temperature is from 50 to 100° C., preferably from 85 to 100° C. The treatment time is from 5 to 60 minutes, preferably from 10 to 30 minutes.

In the mixing step, it is preferable to disperse the aforementioned (A) hydrophobic component and the (B) microorganism in an aqueous solvent, to prepare a mixture of raw materials in a slurry state.

In the present specification, an aqueous solvent refers to water or an aqueous solution containing a water-soluble organic solvent. Examples of the water include tap water, distilled water, ion-exchanged water, and purified water. Examples of the water-soluble organic solvent include lower alcohols such as ethanol. The mixture of raw materials may contain another component than the (A) hydrophobic component which may be contained in a microbial microcapsule described hereafter.

The content of the (A) hydrophobic component in the mixture of raw materials may vary in accordance with the type. From the viewpoint of productivity, it is preferably 11 mass % or more, more preferably 15 mass % or more, further more preferably 20 mass % or more, and is preferably 80 mass % or less, more preferably 70 mass % or less, and further more preferably 60 mass % or less. The content of the (A) hydrophobic component in the mixture of raw materials is preferably from 11 to 80 mass %, more preferably from 15 to 70 mass %, and even more preferably from 20 to 60 mass %.

The content of the microorganism (B) in the mixture of raw materials is preferably 5 mass % or more, more preferably 7 mass % or more, and further more preferably 10 mass % or more, as a dry mass, from the viewpoint of productivity, and is preferably 30 mass % or less, more preferably 25 mass % or less, and further more preferably 20 mass % or less, from the viewpoint of working efficiency such as stirring and separating operation. Then, the content of the (B) microorganism in the mixture of raw materials is preferably from 5 to 30 mass %, more preferably from 7 to 25 mass %, and further more preferably from 10 to 20 mass %. In the present specification, the dry mass of the (B) microorganism refers to a residue obtained by drying the microorganism in a dryer at 105° C. for 12 hours to remove volatile substances.

In the mixing step, mass ratio of the (A) hydrophobic component to the dry mass of the microorganism (B), [(A)/(B)], is above 2, preferably 2.5 or more, more preferably 3.0 or more, further more preferably 4 or more, and even more preferably 5 or more, from the viewpoint of improving the encapsulation percentage of the (A) hydrophobic component, and is preferably 8 or less, more preferably 7 or less, and even more preferably 6 or less, from the viewpoint of productivity. Then, the mass ratio of the (A) hydrophobic component to the dry mass of the (B) microorganism, [(A)/(B)], is above 2, preferably above 2 and 8 or less, more preferably from 2.5 to 7, further more preferably from 2.5 to 8, further more preferably from 3.0 to 7, further more preferably from 4 to 7, and even more preferably from 5 to 6.

The temperature in the mixing step is preferably from 20 to 80° C., more preferably from 25 to 60° C., further more preferably from 30 to 60° C., and even more preferably from 35 to 50° C., from the viewpoint of improving the encapsulation percentage of the (A) hydrophobic component.

The mixing time is preferably 3 hours or more, more preferably 5 hours or more, further more preferably 10 hours or more, and even more preferably 15 hours or more, from the viewpoint of improving the encapsulation percentage of the (A) hydrophobic component, and is also preferably 72 hours or less, more preferably 48 hours or less, and even more preferably 24 hours or less, from the viewpoint of productivity. Then, the mixing time is preferably from 3 to 72 hours, more preferably from 5 to 48 hours, further more preferably from 10 to 48 hours, and further more preferably from 15 to 24 hours.

The agitation condition in the mixing step can be appropriately adjusted. From the viewpoint of improving the encapsulation percentage of the (A) hydrophobic component, the agitation condition is preferably greater than 0 r/min, more preferably 50 r/min or more, even more preferably 100 r/min or more, and is preferably 300 r/min or less, more preferably 250 r/min or less, and even more preferably 200 r/min or less. The agitation condition is preferably greater than 0 r/min and less than or equal to 300 r/min, more preferably from 50 to 250 r/min, and even more preferably from 100 to 200 r/min. Here, in the present specification, the agitation condition refers to the number of revolutions in reciprocating shake.

By such a mixing step, the (A) hydrophobic component can be encapsulated in the microorganism (B). After the mixing step, the microbial microcapsules can be separated by a separation operation such as centrifugation or filtration. The separated microbial microcapsules may be washed, dried, or the like, if necessary.

The microbial microcapsules obtained by the method of the present invention have a higher encapsulation percentage of the (A) hydrophobic components. The encapsulation percentage of the preferred (A) hydrophobic component is as described hereinafter.

Therefore, the microbial microcapsule containing the (A) hydrophobic component of the present invention may be used for various products such as pharmaceuticals, quasi-drugs, cosmetics, foods, and agricultural chemicals. In particular, it can be suitably utilized for, for example, a pest control agent against a sanitary pest or an agricultural pest by taking advantage of the edible property of the pest.

### [Microbial Microcapsules]

The microbial microcapsule of the present invention contains (A) a hydrophobic component having a surface tension above 33.6 mN/m at 25° C. encapsulated in (B) a microorganism, and the encapsulation percentage defined by the following equation (1) is above 54 mass %. It is preferable that the encapsulation percentage be high, from the viewpoint of efficient use of the (A) hydrophobic component. The encapsulation percentage is preferably 55 mass % or more, more preferably 57 mass % or more, further more preferably 60 mass % or more, and even more preferably 64 mass % or more.

Encapsulation percentage (mass %)=[mass of (A) hydrophobic component/(mass of (A) hydrophobic component+dry mass of microorganism (B)]×100 (1)

Setting the encapsulation percentage of the hydrophobic component high can serve to provide a microbial capsule containing a high amount of other various hydrophobic active components.

In the microbial microcapsules of the present invention, the (A) hydrophobic components having a surface tension above 33.6 mN/m at 25° C. and the (B) microorganism are as described hereinbefore.

The microbial microcapsule of the present invention may contain, not only the (A) hydrophobic component, but also a solvent, a surfactant, a stabilizer, a pH adjuster, a saccharide, a salt, a perfume, a pigment, and the like as appropriate, as long as they do not interfere with the efficacy of the present invention.

From the viewpoint of improving the encapsulation percentage of the hydrophobic component, it is preferable that the method for producing the microbial microcapsule of the present invention is preferably as the following constitutions [1] to [4].
[1] A method for producing a microbial microcapsule, comprising mixing (A) a hydrophobic component having a surface tension at 25° C. of above 33.6 mN/m and equal to 72 mN/m or less and (B) a microorganism, wherein the mixing is carried out under a condition of mass ratio of the (A) hydrophobic component to the dry mass of the microorganism (B), [(A)/(B)], being above 2 and 8 or less.
[2] A method for producing a microbial microcapsule comprising mixing (A) a hydrophobic component having a surface tension of from 35.1 to 38.5 mN/m at 25° C. and (B) a microorganism, wherein the mixing is carried out under a condition that mass ratio of the (A) hydrophobic component to the dry mass of the (B) microorganism, [(A)/(B)], being from 2.5 to 7.
[3] A method for producing a microbial microcapsule, comprising mixing (A) a hydrophobic component having a surface tension above 33.6 mN/m and 72 mN/m or less, and (B) a microorganism, wherein the content of the (A) hydrophobic component in the mixed raw materials is from 11 to 80 mass %, and the content of the microorganism (B) in the mixed raw materials is from 5 to 30 mass %, and the mixing is carried out under a condition of mass ratio of the (A) hydrophobic component to the dry mass of the microorganism (B), [(A)/(B)], being above 2 and 8 or less.
[4] A method for producing a microbial microcapsule, comprising mixing (A) a hydrophobic component having a surface tension of from 35.1 to 38.5 mN/m at 25° C. and (B) a microorganism, wherein the content of the (A) hydrophobic component in the mixture of raw materials is from 11 to 80 mass %, and the content of the microorganism (B) in the mixture of raw materials is from 5 to 30 mass %, and the mixing is carried out under a condition of mass ratio of the (A) hydrophobic component to the dry mass of the microorganism (B), [(A)/(B)], being from 2.5 to 7.

It is preferable that the microbial microcapsule of the present invention have the following configurations [5] to [8], from the viewpoint of improving the encapsulation percentage of the hydrophobic components.
[5] A microorganism microcapsule comprising (A) a hydrophobic component having a surface tension above 33.6 mN/m and 72 mN/m or less at 25° C. encapsulated in (B) a microorganism, wherein the encapsulation percentage defined by the following equation (1) is 55 mass % or more.

Encapsulation percentage (mass %)=[mass of (A) hydrophobic component/(mass of (A) hydrophobic component+dry mass of (B) microorganism)]×100 (1)
[6] A microbial microcapsule comprising (A) a hydrophobic component having a surface tension of from 35.1 to 38.5 mN/m at 25° C. encapsulated in (B) a microorganism, wherein the encapsulation percentage defined by the following equation (1) is 57 mass % or more.

Encapsulation percentage (mass %)=[mass of (A) hydrophobic component/(mass of (A) hydrophobic component+dry mass of (B) microorganism)]×100 (1)
[7] A microbial microcapsule comprising (A) a hydrophobic component having a surface tension above 33.6 mN/m and 72 mN/m or less at 25° C. in (B) a yeast, wherein the encapsulation percentage defined by the following equation (1) is 55 mass % or more.

Encapsulation percentage (mass %)=[mass of (A) hydrophobic component/(mass of (A) hydrophobic component+dry mass of (B) microorganism)]×100 (1)
[8] A microbial microcapsule comprising (A) a hydrophobic component having a surface tension of from 35.1 to 38.5 mN/m at 25° C. encapsulated in (B) a yeast, wherein the encapsulation percentage defined by the following equation (1) is 57 mass % or more.

Encapsulation percentage (mass %)=[mass of (A) hydrophobic component/(mass of (A) hydrophobic component+dry mass of (B) microorganism)]×100 (1)

### EXAMPLES

### <Calculation Method of Encapsulation Percentage of (A) Hydrophobic Component>

The slurry (1 mL) of yeast microcapsules or Nannochloropsis microcapsules was centrifuged (CF15RX manufactured by HITACHI, 15 000 r/min, 1 min). Therefrom, a supernatant water was removed. Thereto were added methanol (0.5 mL) and chloroform (0.25 mL), and the mixture was resuspended, and was allowed to stand for 10 minutes. Then chloroform (0.5 mL) and distilled water (0.25 mL) were further added thereto, and a capsule inclusion was extracted. After centrifugation (CF15RX manufactured by HITACHI, 15 000 r/min, 1 min), a lower oil layer was collected, and its content (amount of the (A) hydrophobic component) was calculated by gas chromatography analysis or high-performance liquid chromatography analysis. The encapsulation percentage of the (A) hydrophobic component was calculated by the following equation.

Encapsulation percentage (mass)=[mass of (A) hydrophobic component/(mass of (A) hydrophobic component+dry mass of (B) microorganism (yeast or Nannochloropsis)]×100

### <Determination of Surface Tension>

The surface tension of the (A) hydrophobic components was measured by the capillary rise method at 25° C. and atmospheric pressure by using DG-1 manufactured by Surfgauge INSTRUMENTS. Since this liquid height is determined on the basis of the density of water, it needs to be corrected by density. The density was measured by a portable density hydrometer DA-130N manufactured by Kyoto Electronics Manufacturing Co., Ltd.

### Examples 1 to 9 and Comparative Examples 1 to 18

In the examples, yeast refers to Saccharomyces cerevisiae. A residual (trade name: Yeast Wrap, manufactured by Mitsubishi Corporation Life Sciences Limited) obtained by subjecting yeast to a treatment for eluting its intrayeast component was dispersed in distilled water so as to have 5 mass % (dry mass) and 5 to 30 mass % of the hydrophobic component set forth in Table 1 to give a mixture of raw material slurry. Mass ratios of hydrophobic components to dry mass of the yeast [(A)/(B)] are shown in Table 1.

This mixture slurry of raw materials was shaken for 17 hours with a 200 r/min in a reciprocating shaker at 40° C., to give an encapsulated yeast slurry. From the obtained encapsulated yeast slurry, the encapsulated yeast was precipitated by centrifugation (CF15RX manufactured by HITACHI, 15 000 r/min, 1 min), and the supernatant containing the unutilized (A) hydrophobic component was removed and washed 2 times with the same amount of distilled water to give yeast microcapsules. Encapsulation percentage of (A) hydrophobic component of the yeast microcapsules was calculated.

The conditions of the examples and comparative examples and the encapsulation percentage of the (A) hydrophobic component are shown in Table 1.

TABLE 1

| | | | Examples | | | | | | | | |
|---|---|---|---|---|---|---|---|---|---|---|---|
| | | | 1 | 2 | 3 | 4 | 5 | 6 | 7 | 8 | 9 |
| Mixing | Hydrophobic component | | Carvacrol | | | Methyl salicylate | | | Benzyl alcohol | | |
| step | (A) | | | | | | | | | | |
| | Surface | [mN/m] | 35.1 | | | 36.3 | | | 38.5 | | |
| | tension in | | | | | | | | | | |
| | (A) | | | | | | | | | | |
| | mass ratio | [g_oil/g_ | 3 | 4 | 6 | 3 | 4 | 6 | 3 | 4 | 6 |
| | [(A)/(B)] | yeast] | | | | | | | | | |
| Results | Encapsulation | [Mass %] | 65 | 68 | 68 | 72 | 73 | 74 | 75 | 84 | 86 |
| | percentage | | | | | | | | | | |

| | | | Comparison Examples | | | | | | | | | | | | | | | | | |
|---|---|---|---|---|---|---|---|---|---|---|---|---|---|---|---|---|---|---|---|---|
| | | | 1 | 2 | 3 | 4 | 5 | 6 | 7 | 8 | 9 | 10 | 11 | 12 | 13 | 14 | 15 | 16 | 17 | 18 |
| Mix- | Hydrophobic | | Toluene | | | Caryo- | | Geraniol | | Oleic acid | | | Farnesene | | | L- | | Car- | Methyl | Benzyl |
| ing | component (A) | | | | | phyllene | | | | | | | | | | carboxyl | | vacrol | salicy- | alcohol |
| step | | | | | | | | | | | | | | | | | | | late | |
| | Surface | [mN/ | 26.8 | | | 29.5 | | 29.8 | | 31.1 | | | 31.5 | | | 33.6 | | 35.1 | 36.3 | 38.5 |
| | tension | m] | | | | | | | | | | | | | | | | | | |
| | in (A) | | | | | | | | | | | | | | | | | | | |
| | mass | [g_oil/ | 1 | 2 | 4 | 2 | 4 | 2 | 4 | 1 | 2 | 4 | 1 | 2 | 4 | 2 | 4 | 2 | 2 | 2 |
| | ratio | g_ | | | | | | | | | | | | | | | | | | |
| | [(A)/(B)] | yeast] | | | | | | | | | | | | | | | | | | |
| Re- | Encap- | [Mass | 3.1 | 3.5 | 7.9 | 9.4 | 9.8 | 25 | 25 | 28 | 28 | 28 | 25 | 25 | 25 | 54 | 54 | 53 | 53 | 53 |
| sults | sulation | %] | | | | | | | | | | | | | | | | | | |
| | percent- | | | | | | | | | | | | | | | | | | | |
| | age | | | | | | | | | | | | | | | | | | | |

### Examples 10 to 18 and Comparative Examples 19 to 35

In the examples, Nannochloropsis refers to Nannochloropsis sp. Each of product obtained by spray-drying Nannochloropsis (trade name: Smebenanno W, manufactured by Smebe Japan Co., Ltd.) and hydrophobic component set forth in Table 2 was dispersed in distilled water so as to have 5 mass % (dry mass) and 5 to 30 mass %, respectively to give a mixture slurry of raw materials. Mass ratios of hydrophobic component to dry mass of Nannochloropsis, [(A)/(B)], are shown in Table 2.

The mixture slurry of raw materials was shaken for 17 hours with a 200 r/min in a reciprocating shaker at 40° C., to give an encapsulated Nannochloropsis slurry. From the obtained encapsulated Nannochloropsis slurry, the encapsulated Nannochloropsis was precipitated by centrifugation (CF15RX manufactured by HITACHI, 15 000 r/min, 10 min), and the supernatant containing the unutilized (A) hydrophobic component was removed and washed 2 times with the same amount of distilled water to give a Nannochloropsis microcapsule. The encapsulation percentage of the (A) hydrophobic component of the Nannochloropsis microcapsules was calculated.

The condition of the examples and comparative examples and the encapsulation percentage of the (A) hydrophobic component are shown in Table 2.

TABLE 2

| | | | Examples | | | | | | | | |
|---|---|---|---|---|---|---|---|---|---|---|---|
| | | | 10 | 11 | 12 | 13 | 14 | 15 | 16 | 17 | 18 |
| Mixing | Hydrophobic component | | Carvacrol | | | Methyl salicylate | | | Benzyl alcohol | | |
| step | (A) | | | | | | | | | | |
| | Surface | [mN/m] | 35.1 | | | 36.3 | | | 38.5 | | |
| | tension in (A) | | | | | | | | | | |
| | mass ratio | [g_oil/g_ | 3 | 4 | 6 | 3 | 4 | 6 | 3 | 4 | 6 |
| | [(A)/(B)] | yeast] | | | | | | | | | |
| Results | Encapsulation | [Mass %] | 56 | 60 | 62 | 59 | 62 | 64 | 78 | 80 | 82 |
| | percentage | | | | | | | | | | |

| | | | Comparison Examples | | | | | | | | | | | | | | | | |
|---|---|---|---|---|---|---|---|---|---|---|---|---|---|---|---|---|---|---|---|
| | | | 19 | 20 | 21 | 22 | 23 | 24 | 25 | 26 | 27 | 28 | 29 | 30 | 31 | 32 | 33 | 34 | 35 |
| Mix- | Hydrophobic | | Toluene | | | Caryo- | | Geraniol | | Oleic | | Farnesene | | | L- | | Car- | Methyl | Benzyl |
| ing | component (A) | | | | | phyllene | | | | acid | | | | | carboxyl | | vacrol | salicy- | alcohol |
| step | | | | | | | | | | | | | | | | | | late | |
| | Surface | [mN/ | 26.8 | | | 29.5 | | 29.8 | | 31.1 | | 31.5 | | | 33.6 | | 35.1 | 36.3 | 38.5 |
| | tension | m] | | | | | | | | | | | | | | | | | |
| | in (A) | | | | | | | | | | | | | | | | | | |
| | mass | [g_oil/ | 1 | 2 | 4 | 2 | 4 | 2 | 4 | 2 | 4 | 1 | 2 | 4 | 2 | 4 | 2 | 2 | 2 |
| | ratio | g_ | | | | | | | | | | | | | | | | | |
| | [(A)/(B)] | yeast] | | | | | | | | | | | | | | | | | |
| Re- | Encap- | [Mass | 6.7 | 2.4 | 3.1 | 22 | 32 | 24 | 35 | 25 | 20 | 19 | 27 | 28 | 34 | 37 | 52 | 54 | 53 |
| sults | sulation | %] | | | | | | | | | | | | | | | | | |
| | percent- | | | | | | | | | | | | | | | | | | |
| | age | | | | | | | | | | | | | | | | | | |

As is evident from Tables 1 and 2, it was confirmed that by using a hydrophobic component having a surface tension above 33.6 mN/m at 25° C., and mixing the microorganism and the hydrophobic component such that mass ratio of the hydrophobic component to the dry mass of the microorganism is above 2, a microbial microcapsule containing a hydrophobic component in a high encapsulation percentage can be obtained.

1. A method for producing a microbial microcapsule, comprising: mixing a (A) hydrophobic component having a surface tension above 33.6 mN/m at 25° C. and a microorganism (B) for 3 hours or more,
wherein the mixing is carried out under a condition that a mass ratio of the (A) hydrophobic component to a dry mass of the microorganism (B), [(A)/(B)], is above 2.

2. The method for producing a microbial microcapsule according to claim 1, wherein the (A) hydrophobic component has a log P value of 1.0 or more.

3. The method for producing a microbial microcapsule according to claim 1, wherein the mixing is performed at from 20 to 80° C.

4. The method for producing a microbial microcapsule according to claim 1, wherein the (B) microorganism has a cell wall.

5. The method for producing a microbial microcapsule according to claim 1, wherein the microorganism (B) is a yeast.

6. The method for producing a microbial microcapsule according to claim 1, wherein the (B) microorganism is a fine alga.

7. A microbial microcapsule encapsulating a (A) hydrophobic component having a surface tension above 33.6 mN/m at 25° C. in a microorganism (B), wherein an encapsulation percentage defined by the following equation (1) is above 60 mass %,
Encapsulation percentage (mass %)=[mass of (A) hydrophobic component/(mass of (A) hydrophobic component+dry mass of (B) microorganism)]×100 (1)

8. The microbial microcapsule according to claim 7, wherein the (A) hydrophobic component has a log P value of equal to 1.0 or more.

9. The microorganism microcapsule according to claim 7, wherein the microorganism (B) has a cell wall.

10. A microbial microcapsule according to claim 7, wherein the microorganism (B) is a yeast.

11. The microorganism microcapsule according to claim 7, wherein the microorganism (B) is a fine alga.

12. The method for producing a microbial microcapsule according to claim 1, wherein the (A) hydrophobic component has a surface tension of from 35.1 to 38.5 mN/m at 25° C.

13. The method for producing a microbial microcapsule according to claim 1, wherein
a content of the (A) hydrophobic component in a mixture of raw materials containing the (A) and the (B) is from 11 to 80 mass %,
a content of the microorganism (B) in the mixture is from 5 to 30 mass %, and
the mixing is carried out under a condition that a mass ratio of the (A) hydrophobic component to the dry mass of the microorganism (B), [(A)/(B)], is from 2.5 to 7.

14. The method for producing a microbial microcapsule according to claim 1, wherein the (A) hydrophobic component and the (B) microorganism are mixed for 10 hours or more.

15. A method for producing a microbial microcapsule, comprising:
mixing (A) a hydrophobic component having a surface tension of from 35.1 to 38.5 mN/m at 25° C. and (B) a microorganism for 3 hours or more,
wherein a content of the (A) hydrophobic component in a mixture of raw materials is from 11 to 80 mass %,
a content of the microorganism (B) in the mixture of raw materials is from 5 to 30 mass %, and
the mixing is carried out under a condition that a mass ratio of the (A) hydrophobic component to a dry mass of the microorganism (B), [(A)/(B)], is from 2.5 to 7.

16. The method for producing a microbial microcapsule according to claim 15, wherein the mixing is performed at from 20 to 80° C.

17. The method for producing a microbial microcapsule according to claim 15, wherein the (B) microorganism has a cell wall.

18. The method for producing a microbial microcapsule according to claim 15, wherein the microorganism (B) is a yeast.

19. The method for producing a microbial microcapsule according to claim 15, wherein the (B) microorganism is a fine alga.

20. The method for producing a microbial microcapsule according to claim 7, wherein the (A) hydrophobic component has a surface tension of from 35.1 to 38.5 mN/m at 25° C.
